(19) United States
(12) Patent Application Publication
(10) Pub. No.: US 2002/0133110 A1
(43) Pub. Date: Sep. 19, 2002

(54) Device for treatment of inflamed tissue

(76) Inventor: Jonathan Stuart Citow, Lake Forest, IL (US)

Correspondence Address: Russell L. McI lwain, Jones, Day, Reavis & Pogue, 77 West Wacker, Chicago, IL, 60601, US

(21) Appl. No.: 09/810,295

(22) Filed: Mar. 16, 2001

Related U.S. Application Data
(60) Provisional application No. 60/262,954, filed on Jan. 19, 2001.

(51) Int. Cl. A61M 35/00

(52) U.S. Cl. 604/1; 604/289

(57) ABSTRACT

A double-ended plastic handle is fitted with a cotton swab at each end. Preferably, the swabs are treated with a topical solution of Lidocaine Hydrochloride and each device is wrapped individually in a plastic enclosure. The device may be easily unwrapped and the swabs may be used to conveniently administer the topical solution to affected tissue without requiring any clean-up after discarding the used device.

### CROSS-REFERENCE TO RELATED APPLICATION

Applicant claims priority from U.S. Provisional Application Serial No. 60/262,954, filed Jan. 19, 2001.

### BACKGROUND OF THE INVENTION

1. Field of the Invention

The present invention relates generally to the medicinal treatment of inflamed tissue and, more particularly to a disposable device for convenient and sanitary topical application of suitable compounds for alleviating the symptoms of hemorrhoid irritation and other anorectal diseases.

2. Description of the Related Art

Many chemical compounds are disclosed in the literature for treatment of inflamed hemorrhoidal tissue and other anorectal diseases. These compounds have the benefit of reducing itching, soreness and other side effects associated with inflamed anorectal tissue. Examples of such compounds include zinc peroxide, as disclosed in U.S. Pat. No. 2,436,673, and urea hydrogen peroxide or benzyl peroxide in combination with hydrocortisone, as disclosed in U.S. Pat. No. 4,514,384, to name a few. Typically, these preparations are provided in the form of suppositories having a semi-solid consistency at room temperature such that they can be wrapped in foil and administered manually.

A disadvantage of common preparations sold in suppository form for the treatment of hemorrhoids or other anorectal conditions is that they melt when their temperature is raised, such as by grasping them with the fingers, thereby leaving residue on the fingers which the user typically must remove as by hand washing with soap and water, for example. Alternatively, the user might employ a surgical glove to administer the suppository then discard the glove when the process in complete. In either event, the process of administering a suppository of the type commonly used to treat inflamed hemorrhoidal tissue can be an inconvenient and time consuming process.

### SUMMARY OF THE INVENTION

The present invention improves over the prior art by providing a double-ended plastic handle fitted with a cotton swab at each end. Preferably, the swabs are treated with a topical solution of Lidocaine Hydrochloride and each device is wrapped individually in a plastic enclosure. The device may be easily unwrapped and the swabs may be used to conveniently administer the topical solution to the affected tissue without requiring any clean-up after discarding the used device.

### BRIEF DESCRIPTION OF THE DRAWING

The foregoing and other novel features and advantages of the invention will be better understood upon a reading of the following detailed description taken in conjunction with the accompanying drawing wherein:

FIG. 1 is an elevational view of a device according to the invention for administering a topical preparation and shown as wrapped in a suitable plastic enclosure.

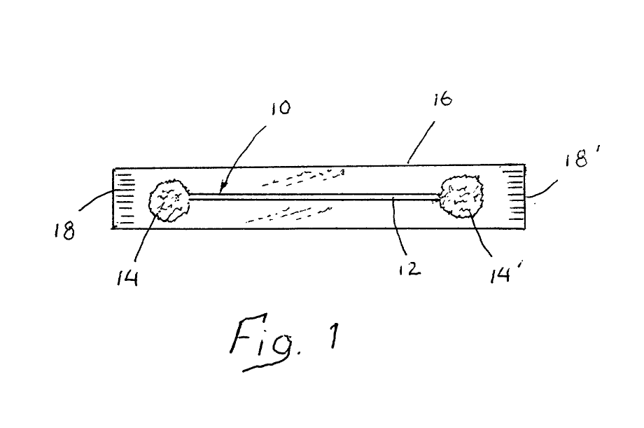

### DETAILED DESCRIPTION OF THE PREFERRED EMBODIMENTS

Referring now to the drawing, there is illustrated an applicator device designated generally by the reference numeral 10, for administering a topical preparation to inflamed hemorrhoidal tissue, for example. The device includes as its principal components a stick-like handle 12 preferably constructed of a break-resistant plastic and having two opposed ends to which cotton swabs 14 and 14′ are attached such as by a suitable adhesive. The device 10 is shown as being enclosed in a suitable plastic wrapper 16 having ends 18 and 18′ that are preferably heat-sealed closed. The ends may be conveniently torn open by the user to gain access to the applicator device 10.

It can be appreciated that an applicator device 10 according to the invention offers considerable convenience in the administering of topical preparations, particularly to inflamed hemorrhoidal tissue, and offers considerable advantages over the use of suppositories, for example. The device 10 can very easily be removed from its wrapper 16 and grasped by the handle 12 to apply the preparation without exposing the user's fingers to the preparation. Once the application process is completed, the device 10 may simply be discarded without the need for any clean-up.

Preferably, the swabs 14 and 14′ are saturated with a therapeutic preparation comprising a lidocaine compound, such as a topical solution of about 4% Lidocaine Hydrochloride; a cream, gel, or ointment may also be used. This preparation may also be mixed with anti-inflammatories such as steroids. The handle 12 itself may be on the order of 5 cm long so as to be convenient to carry the device 10 in a purse or wallet.

While the present invention has been described in connection with preferred embodiments, it will be apparent to those skilled in the art that many changes and modifications may be made without departing from the true spirit and scope of the invention. Accordingly, it is intended by the appended claims to cover all such changes and modifications which come within the spirit and scope of the invention.

What is claimed is:

1. A device for treatment of inflamed hemorrhoidal tissue or anorectal disease comprising:
a thin generally elongate handle having two opposed ends with at least one end having a swab affixed thereto;
said swab being saturated with a therapeutic preparation for said treatment of said tissue or disease;
said handle with said saturated swab being enclosed in a tearable wrapper;
wherein said wrapper can be manually torn open to remove said swab and handle to apply said preparation to an anorectal area.

2. The device of claim 1 wherein each end of said handle is provided with a swab.

3. The device of claim 1 wherein said handle is formed from a break-resistant plastic.

4. The device of claim 1 wherein said wrapper is provided with heat-sealed ends.

5. The device of claim 1 wherein said swab is formed of cotton.

6. The device of claim 1 wherein said preparation includes a lidocaine compound.

7. The device of claim 1 wherein said preparation includes a topical solution of about 4% of a lidocaine compound.

8. The device of claim 1 wherein said handle is approximately 5 cm in length.

9. A prepackaged device for treatment of inflamed hemorrhoidal tissue or anorectal disease comprising:
a thin generally elongate stick-like handle having opposed ends provided with a cotton swab affixed to each end;
said swabs being saturated in a therapeutic preparation for said treatment;
said handle and swab assembly being enclosed in a heat-sealed tearable plastic wrapper which can be manually opened to remove said assembly therefrom;
wherein said preparation comprises a lidocaine compound.
